(19) United States
(12) Patent Application Publication
(10) Pub. No.: US 2023/0062963 A1
(43) Pub. Date: Mar. 2, 2023

(54) METHOD OF PRODUCING 1,3-BUTADIENE

(71) Applicants: ENEOS CORPORATION, Chiyoda-ku (JP); ENEOS MATERIALS CORPORATION, Tokyo (JP)

(72) Inventors: Naoko URUSHIZAKO, Chiyoda-ku (JP); Takashi MORI, Minato-Ku (JP)

(73) Assignees: ENEOS CORPORATION, Chiyoda-ku (JP); ENEOS MATERIALS CORPORATION, Tokyo (JP)

(21) Appl. No.: 17/904,391

(22) PCT Filed: Feb. 22, 2021
(86) PCT No.: PCT/JP2021/006516

§ 371 Date: Aug. 17, 2022

(30) Foreign Application Priority Data
Feb. 25, 2020 (JP) 2020-029511

(51) Int. Cl. C07C 5/48; B01J 23/887

(52) U.S. Cl. CPC C07C 5/48; B01J 23/8876; C07C 2523/887

(57) ABSTRACT

A method of producing 1,3-butadiene including feeding oxygen and a feedstock gas containing n-butene into a reactor from the lower section of the reactor provided with a metal atom-containing catalyst, so that a product gas containing 1,3-butadiene is obtained through oxidative dehydrogenation of n-butene; cooling the product gas containing the 1,3-butadiene; and separating the 1,3-butadiene from the cooled product gas through selective absorption into an absorption solvent.

### TECHNICAL FIELD

One embodiment of the present invention relates to a method of producing 1,3-butadiene, and more particularly to a method of producing 1,3-butadiene with use of oxidative dehydrogenation reaction.

### BACKGROUND ART

Conventionally, as a method of producing 1,3-butadiene (hereinafter also simply referred to as “butadiene”), there has been used a method in which components other than butadiene is separated from a fraction having 4 carbon atoms (hereinafter also referred to as “C4 fraction”), which is yielded from naphtha cracking, by distillation.

The demand for butadiene as a feedstock such as for synthetic rubber is increasing, on the other hand, the supply of C4 fraction is decreasing under circumstances such as the production method of ethylene being shifting from naphtha cracking to ethane pyrolysis, and hence there is a demand for a method of producing butadiene without using C4 fraction as the feedstock.

In view of this, as a method of producing butadiene, much focus is being placed on a method of obtaining butadiene as separated from a product gas yielded from oxidative dehydrogenation of n-butene. For this production method, there has been studied a method in which, in order to improve the separation efficiency of butadiene, the pressure of the product gas from oxidative dehydrogenation reaction is increased, thereafter butadiene is separated by using an absorption solvent (see Patent Literatures 1 to 5.).

### CITATION LIST

### Patent Literature

Patent Literature 1: JP 5621304 B2

Patent Literature 2: JP 5652151 B2

Patent Literature 3: JP 5714857 B2

Patent Literature 4: JP 2012-111751 A

Patent Literature 5: JP 2016-500333 A

### SUMMARY OF INVENTION

### Technical Problem

In the conventional method of producing butadiene as described above, typically, a catalyst layer is provided in the lower section of a reactor where the oxidative dehydrogenation reaction takes place, a feedstock gas is fed from the upper section of the reactor, and a product gas is discharged from the lower section of the reactor. As a result of a great deal of studies made by the present inventors on the method of producing 1,3-butadiene with use of oxidative dehydrogenation reaction, it has been revealed that a fouling (by-product) due to a side reaction involved in the reaction is accumulated in the catalyst layer provided in the lower section of the reactor where the oxidative dehydrogenation reaction takes place. For this reason, the entirety of the catalyst needs to be pulled out from the lower section of the reactor for the replacement of the contaminated catalyst.

An embodiment of the present invention has been made based on the stated circumstances, and provides a method of producing 1,3-butadiene with which the working efficiency involved in the catalyst replacement is improved.

### Solution to Problem

The present inventors have found that the above-described problem can be solved by the method of producing 1,3-butadiene set forth below, and have completed the present invention. The present invention relates to, for example, the following [1] to [5].

[1] A method of producing 1,3-butadiene, including: a step A in which oxygen and a feedstock gas that contains n-butene are fed into a reactor from a lower section of the reactor provided with a metal atom-containing catalyst so that a product gas that contains 1,3-butadiene is obtained through oxidative dehydrogenation of n-butene; a step B of cooling the product gas that contains 1,3-butadiene; and a step C in which 1,3-butadiene is separated from the cooled product gas that contains 1,3-butadiene through selective absorption into an absorption solvent.
[2] The method of producing 1,3-butadiene according to the [1], wherein the reactor to be used in the step A is a shell-and-tube heat exchanger reactor that includes a plurality of reaction tubes in which a metal atom-containing catalyst is filled.
[3] The method of producing 1,3-butadiene according to the [1] or [2], wherein the metal atom-containing catalyst is a composite oxide catalyst that contains at least molybdenum and cobalt.
[4] The method of producing 1,3-butadiene according to any of the [1] to [3], wherein the feedstock gas in the step A contains n-butene at a concentration of 40% by volume or higher.

[5] The method of producing 1,3-butadiene according to any of the [1] to [4], wherein the step A is held at a pressure of 0 to 0.4 MPaG, and the steps A to C are held at an equal pressure, or the step A, the step B, and the step C each are held at a pressure decreasing in this order.

### Advantageous Effects of Invention

According to the method of producing 1,3-butadiene according to an embodiment of the present invention, a feedstock gas is fed from the lower section of a reactor and discharged from the upper section thereof, whereby a fouling due to a by-product in a catalyst layer is generated in the upper layer portion of the catalyst layer, so that only the contaminated catalyst can be removed from the upper layer portion so as to be replaced. An uncontaminated catalyst between the middle layer portion and the lower layer portion, for example, in the catalyst layer can be continuously used, so that only a small amount of the catalyst needs to be replaced, and hence the working time and the catalyst cost can be reduced.

### BRIEF DESCRIPTION OF DRAWING

FIG. 1 is a flowchart that illustrates an example of a specific method for implementing a method of producing 1,3-butadiene according to an embodiment of the present invention.

### DESCRIPTION OF EMBODIMENTS

Hereinafter, the embodiments of the present invention will be described.

A method of producing 1,3-butadiene according to an embodiment of the present invention, includes:

a step A in which oxygen and a feedstock gas that contains n-butene are fed into a reactor from the lower section of the reactor provided with a metal atom-containing catalyst, so that a product gas that contains 1,3-butadiene is obtained through oxidative dehydrogenation of n-butene;

a step B of cooling the product gas that contains 1,3-butadiene; and

a step C in which 1,3-butadiene is separated from the cooled product gas that contains 1,3-butadiene through selective absorption into an absorption solvent.

As used herein, “n-butene” includes 1-butene and 2-butene. Also, “2-butene” includes cis-2-butene and trans-2-butene. The “product gas that contains 1,3-butadiene” obtained in the step A and is discharged out of the reactor is also simply referred to as a “product gas”.

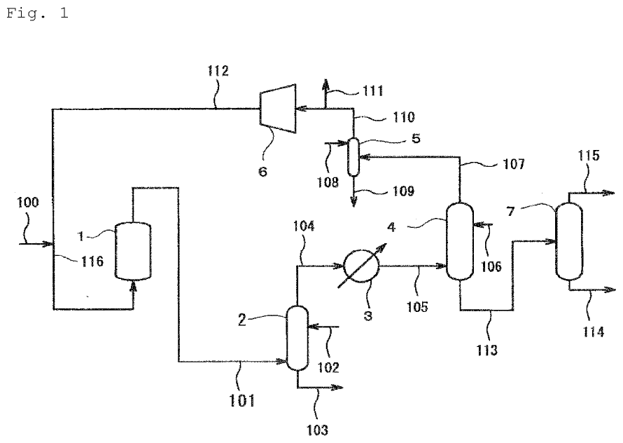

Hereinafter, there will be described a specific example of a method of producing butadiene according to an embodiment of the present invention in detail with reference to FIG. 1, but the present invention will not be limited by the description related to FIG. 1 in any way. FIG. 1 is a flowchart illustrating an example of a specific method for implementing a method of producing butadiene according to an embodiment of the present invention.

The method of producing butadiene according to an embodiment of the present invention can further include a solvent removing step in which the absorption solvent (hereinafter also referred to as “absorption liquid”) obtained in step C, which has absorbed at least 1,3-butadiene, is subjected to solvent separation process.

In addition, the method of producing butadiene according to an embodiment of the present invention can further include a circulation step in which an effluent such as inert gases separated in the step C, are returned to, that is, sent as a return gas to the step A.

### Step A

In the step A, oxygen and a feedstock gas that contains n-butene are fed to a reactor from the lower section of the reactor provided with a metal atom-containing catalyst, thereby producing a product gas that contains 1,3-butadiene through oxidative dehydrogenation of n-butene. In the step A, the oxidative dehydrogenation reaction between the feedstock gas and oxygen (molecular oxygen) is carried out in a reactor 1 as shown in FIG. 1.

### Reactor

A reactor 1 includes: a gas inlet provided in its lower section, preferably in its bottom; and a gas outlet provided in its upper section, preferably in its top, and is in the form of a tower in which a catalyst layer (not shown) is formed by filling a metal atom-containing catalyst such as a metal oxide catalyst in the reactor, preferably in the upper section of the reactor. The reactor 1 is preferably a shell-and-tube heat exchanger reactor including a plurality of reaction tubes in which a metal atom-containing catalyst is filled. In the reactor 1, a pipe 100 and a pipe 112 are connected to the gas inlet via a pipe 116, and a pipe 101 is connected to the gas outlet.

Specifically describing the step A, a feedstock gas and a molecular oxygen-containing gas, and if necessary, inert gases and water (water vapor) (these are collectively referred to also as “new feed gas”, hereinafter) are heated to approximately 200° C. or higher and 400° C. or lower through a preheater (not shown) disposed between the reactor 1 and the pipe 100, thereafter being fed to the reactor 1 via the pipe 100 communicated with the pipe 116.

Along with the new feed gas supplied via the pipe 100, a return gas from the circulation step, after being heated through the preheater, can be fed to the reactor 1 via the pipe 112 communicated with the pipe 116.

That is, a mixture of the new feed gas and the return gas (hereinafter also referred to as a “mixed gas”), after being heated through the preheater, can be fed to the reactor 1. In this case, the new feed gas and the return gas may be directly fed from separate pipes to the reactor 1, but are preferably fed in a mixed state from the common pipe 116 as shown in FIG. 1. By providing the common pipe 116, the mixed gas containing various components can be supplied in uniformly pre-mixed state to the reactor 1, so that a situation in which a heterogeneous mixed gas partially forms a detonating gas in the reactor 1, for example, can be obviated.

Subsequently, in the reactor 1 where the mixed gas is supplied, butadiene (1,3-butadiene) is produced by the oxidative dehydrogenation reaction between the feedstock gas and the molecular oxygen-containing gas thereby giving a product gas that contains butadiene. The resultant product gas flows out from the gas outlet of the reactor 1 to the pipe 101.

In an embodiment of the present invention, the feedstock gas and molecular oxygen are fed from the lower section of the reactor 1, and the product gas is discharged from the upper section of the reactor 1, whereby the fouling due to by-products in the catalyst layer is generated in the upper layer portion of the catalyst layer, so that only the contaminated catalyst can be removed from the upper layer portion so as to be replaced. As a result of this, the working time for the replacement of the catalyst layer and the catalyst cost can be reduced.

### Feedstock Gas

The feedstock gas contains a gaseous substance obtained through a vaporizer (not shown) where n-butene (1-butene and 2-butene) as a monoolefin having 4 carbon atoms, which is a feedstock for 1,3-butadiene, is gasified.

In the feedstock gas, the ratio of 2-butene to the total of 1-butene and 2-butene is preferably 50% by volume or more, more preferably 70% by volume or more, and still more preferably 85% by volume or more based on 100% by volume of the total of 1-butene and 2-butene.

The feedstock gas is a combustible gas having flammability.

The concentration of n-butene in the feedstock gas is usually 40% by volume or higher, preferably 60% by volume or higher, more preferably 75% by volume or higher, and particularly preferably 95% by volume or higher based on 100% by volume of the feedstock gas.

The feedstock gas may contain any impurities as long as the advantageous effects of the present invention are not impaired. Specific examples of impurities include: branched monoolefins such as i-butene; and saturated hydrocarbons such as propane, n-butane, and i-butane. In addition, the feedstock gas may contain 1,3-butadiene, which is a target product, as an impurity.

The amount of impurities in the feedstock gas is usually 60% by volume or less, preferably 40% by volume or less, more preferably 25% by volume or less, and particularly preferably 5% by volume or less based on 100% by volume of the feedstock gas. When the amount of impurities is excessively large, the reaction rate tends to decrease or the amount of by-products tends to increase due to a decreased concentration of n-butene in the feedstock gas.

As the feedstock gas, there can be used, for example, a fraction (raffinate 2) containing n-butene as its main component obtained by separating butadiene and i-butene from a C4 fraction (fraction having 4 carbon atoms) produced as a by-product in cracking naphtha, or a butene fraction produced by dehydrogenation or oxidative dehydrogenation of n-butane. It is also possible to use gases containing high-purity 1-butene, cis-2-butene and trans-2-butene obtained in dimerization of ethylene, and mixtures thereof. Furthermore, a gas containing a large amount of hydrocarbons having 4 carbon atoms (this may be abbreviated as “FCC-C4” hereinafter), which is obtained from fluid catalytic cracking where a heavy oil fraction obtained by distillation of crude oil in petroleum refining plants, for example, is decomposed in a fluidized bed condition with use of a powdery solid catalyst and then converted into hydrocarbons having low-boiling points, can be used as a feedstock gas as it is, or FCC-C4 from which impurities such as phosphorus are removed can be used as a feedstock gas as well.

### Molecular Oxygen

In an embodiment of the present invention, oxygen (molecular oxygen (O₂)) is used to conduct oxidative dehydrogenation of n-butene. When supplying molecular oxygen to the reactor, a molecular oxygen-containing gas is preferably used. The molecular oxygen-containing gas is usually a gas that contains molecular oxygen (O₂) in an amount of 10% by volume or more, preferably 15% by volume or more, and more preferably 20% by volume or more.

The molecular oxygen-containing gas can contain, together with molecular oxygen, an optional gas such as molecular nitrogen (N₂), argon (Ar), neon (Ne), helium (He), carbon monoxide (CO), carbon dioxide (CO₂), and water (water vapor). In the case of the optional gas being molecular nitrogen, the amount of the optional gas in the molecular oxygen-containing gas is usually 90% by volume or less, preferably 85% by volume or less, and more preferably 80% by volume or less; in the case of the optional gas being a gas other than molecular nitrogen, the amount of the optional gas in the molecular oxygen-containing gas is usually 10% by volume or less, and preferably 1% by volume or less. In a case where the amount of the optional gas is excessively large, there is a concern that a required amount of molecular oxygen is not allowed to coexist with the feedstock gas in the reaction system (inside the reactor 1).

Preferable specific examples of the molecular oxygen-containing gas include air.

### Inert Gases

Inert gases are preferably fed to a reactor together with the feedstock gas and the molecular oxygen-containing gas. By supplying the inert gases to a reactor, the concentrations (relative concentrations) of the feedstock gas and the molecular oxygen can be adjusted such that the mixed gas does not form the detonating gas in the reactor.

Examples of inert gases include molecular nitrogen (N₂), argon (Ar), and carbon dioxide (CO₂). These can be used alone or two or more. Among them, molecular nitrogen is preferable from an economic viewpoint.

### Water (Water Vapor)

Water is preferably fed to the reactor together with the feedstock gas and the molecular oxygen-containing gas. By supplying water to the reactor, the concentrations (relative concentrations) of the feedstock gas and molecular oxygen can be adjusted such that the mixed gas does not form the detonating gas in the reactor, as in the case of the inert gases described above.

### Mixed Gas

Since the mixed gas of the new feed gas and the return gas contains the combustible feedstock gas and molecular oxygen, the composition thereof is generally adjusted such that the concentration of the feedstock gas does not fall within the range of explosion.

Specifically, the composition of the mixed gas at the gas inlet of the reactor 1 is controlled with the monitoring of the flow rate by a flow meter (not shown) that is installed in a pipe (specifically, a pipe (not shown) communicated with the pipe 100; and the pipe 112) through which each gas (specifically, the feedstock gas, the molecular oxygen-containing gas (for example, air); as well as inert gases and water (water vapor) to be used as necessary) composing the mixed gas is supplied to the reactor 1. As an example, the composition of the new feed gas supplied to the reactor 1 via the pipe 100 is controlled depending on the oxygen concentration of the return gas supplied to the reactor 1 via the pipe 112.

Note that, as used in the present specification, the term “range of explosion” refers to a range having a composition with which a mixed gas is ignited in the presence of any ignition source. It is known that a combustible gas cannot be ignited even if an ignition source is present when the concentration of the combustible gas is lower than a certain value, and this value of concentration is referred to as its lower flammability limit. The lower flammability limit is the lower limit of the range of explosion. It is also known that the combustible gas cannot be ignited even if an ignition source is present when the concentration of the combustible gas is higher than a certain value as well, and this value of concentration is referred to as its upper flammability limit. The upper flammability limit is the upper limit of the range of explosion. These values depend on the concentration of molecular oxygen, these values become typically close to each other with decreasing concentration of molecular oxygen, and when the concentration of molecular oxygen becomes a certain value, these values are identical to each other. The concentration of molecular oxygen at this time is referred to as the limiting oxygen concentration. In the mixed gas, when the concentration of molecular oxygen is below the limiting oxygen concentration, the mixed gas cannot be ignited independent of the concentration of the feedstock gas.

Specifically, in view of the productivity of butadiene and the alleviation of a burden on the metal atom-containing catalyst such as a metal oxide catalyst, the concentration of n-butene in the mixed gas is preferably 2% by volume or more and 30% by volume or less, more preferably 3% by volume or more and 25% by volume or less, and particularly preferably 5% by volume or more and 20% by volume or less based on 100% by volume of the mixed gas. When the concentration of n-butene is excessively low, the productivity of butadiene is liable to decrease, and in contrast, when the concentration of n-butene is excessively high, the burden on a metal atom-containing catalyst such as a metal oxide catalyst is liable to increase.

In the mixed gas, the amount (relative amount) of molecular oxygen with respect to 100 parts by volume of the feedstock gas is preferably 50 parts by volume or more and 170 parts by volume or less, and more preferably 70 parts by volume or more and 160 parts by volume or less. When the amount of molecular oxygen in the mixed gas is out of the above-described range, the adjustment of the concentration of molecular oxygen at the gas outlet of the reactor tends to be difficult even when the reaction temperature is adjusted. When the concentration of molecular oxygen at the gas outlet of the reactor cannot be controlled by the reaction temperature, there is a concern that the decomposition of the target product and the occurrence of side reactions in the reactor cannot be suppressed.

In the mixed gas, the amount (relative amount) of molecular nitrogen with respect to 100 parts by volume of the feedstock gas is preferably 400 parts by volume or more and 1800 parts by volume or less, and more preferably 500 parts by volume or more and 1700 parts by volume or less. The amount (relative amount) of water (water vapor) with respect to 100 parts by volume of the feedstock gas is preferably 0 parts by volume or more and 900 parts by volume or less, and more preferably 80 parts by volume or more and 300 parts by volume or less. When the concentration of molecular nitrogen or the concentration of water is excessively high, in any case, the higher the value, the lower the concentration of the feedstock gas, consequently the production efficiency of butadiene tends to decrease. In contrast, when the concentration of molecular nitrogen or the concentration of water is excessively low, in any case, the lower the value, results in that the concentration of the feedstock gas is prone to be within the range of explosion, or the below described heat removal of reaction system is prone to be difficult.

### Metal Atom-Containing Catalyst

The metal atom-containing catalyst is preferably a metal oxide catalyst. The metal oxide catalyst is not particularly limited as long as it functions as an oxidative dehydrogenation catalyst for the feedstock gas, and a known catalyst can be used, and examples thereof include a catalyst containing a metal oxide including, for example, at least molybdenum (Mo), bismuth (Bi), and iron (Fe), and is preferably a composite oxide catalyst containing at least cobalt (Co) together with molybdenum.

The metal atom-containing catalyst can be used singly or two or more.

Preferable specific examples of the metal oxide include a composite metal oxide represented by the following compositional formula (1). Compositional formula (1):

MoₐBi_{b}Fe_{c}X_{d}YₑZ_{f}O_{g}

In the compositional formula (1), X is at least one selected from the group consisting of Ni and Co. Y is at least one selected from the group consisting of Li, Na, K, Rb, Cs, and Tl. Z is at least one selected from the group consisting of Mg, Ca, Ce, Zn, Cr, Sb, As, B, P, and W. When an atomic ratio of each element is independently represented by each of a, b, c, d, e, f, and g, and when a is 12, b is from 0.1 to 8, c is from 0.1 to 20, d is from 0 to 20, e is from 0 to 4, f is from 0 to 2, and g is the number of atoms of the oxygen element required to satisfy the valence of each of the stated components.

The metal oxide catalyst that contains the composite metal oxide represented by the compositional formula (1) is highly active and highly selective as well as excellent in life stability in a method of producing butadiene through oxidative dehydrogenation reaction.

The method of preparing the metal oxide catalyst is not particularly limited, and there can be used a known method such as evaporation drying method, spray drying method, or oxide mixing method with use of a starting material of each element related to a metal oxide as a component of the metal oxide catalyst to be prepared.

The starting material of each of the above elements is not particularly limited, and examples thereof include oxides, nitrates, carbonates, ammonium salts, hydroxides, carboxylates, ammonium carboxylates, ammonium halides, hydrogen acids, and alkoxides of each component element.

The metal oxide catalyst may be used as being supported on an inert carrier. Example species of carriers include silica, alumina, and silicon carbide.

### Oxidative Dehydrogenation Reaction

A preferable way to start up oxidative dehydrogenation reaction in the step A is that: first, starting the supply of the molecular oxygen-containing gas, inert gases, and water (water vapor) to the reactor, and then adjusting the feeds thereof, whereby the concentration of molecular oxygen at the gas inlet of the reactor is controlled so as to be less than or equal to the limiting oxygen concentration; next, starting the supply of the feedstock gas and then increasing the feed of the feedstock gas and the feed of the molecular oxygen-containing gas so that the concentration of the feedstock gas at the gas inlet of the reactor becomes higher than the upper flammability limit.

During increasing the feeds of the feedstock gas and the molecular oxygen-containing gas, the feed of water (water vapor) may be reduced such that the feed of the mixed gas becomes constant. As a result of this, the gas residence time in the pipe and the reactor is kept constant, and the pressure fluctuation in the reactor can be suppressed.

The pressure of the reactor (specifically, the pressure at the gas inlet of the reactor), that is, the pressure of the step A is preferably 0 MPaG or higher and 0.4 MPaG or lower, more preferably 0.02 MPaG or higher and 0.35 MPaG or lower, and still more preferably 0.05 MPaG or more and 0.3 MPaG or lower. By limiting the pressure of the step A within the above-described range, the reaction efficiency in the oxidative dehydrogenation reaction tends to increase.

In the oxidative dehydrogenation reaction, the gas hourly space velocity (GHSV) determined by the following Formula (1) is preferably 500 h⁻¹ or more and 5000 h⁻¹ or less, more preferably 800 h⁻¹ or more and 3000 h⁻¹ or less, and still more preferably 1000 h⁻¹ or more and 2500 h⁻¹ or less. By limiting the GHSV within the above-described range, the reaction efficiency in the oxidative dehydrogenation reaction can be further improved.

GHSV [h⁻¹]=volumetric gas flow rate converted to standard atmospheric pressure condition [Nm³/h]/catalyst layer volume [m³] Formula (1):

In Formula (1), the “catalyst layer volume” refers to the volume (apparent volume) of the entire catalyst layer including voids.

In the oxidative dehydrogenation reaction, the actual volumetric gas hourly space velocity (actual volumetric GHSV) determined by the following Formula (2) is preferably 500 h⁻¹ or more and 3500 h⁻¹ or less, more preferably 600 h⁻¹ or more and 3000 h⁻¹ or less, and still more preferably 700 h⁻¹ or more and 2500 h⁻¹ or less. By limiting the actual volumetric GHSV within the above-described range, the reaction efficiency in the oxidative dehydrogenation reaction can be further improved.

Actual volumetric GHSV [h⁻¹]=actual volumetric gas flow rate [m³/h]/catalyst layer volume [m³] Formula (2):

In Formula (2), the “catalyst layer volume” refers to the volume (apparent volume) of the entire catalyst layer including voids as in Formula (1).

In the oxidative dehydrogenation reaction, the temperature of the reaction system increases since the oxidative dehydrogenation reaction is an exothermic reaction, and multiple kinds of by-products can be generated. As by-products, unsaturated carbonyl compounds having 3 to 4 carbon atoms such as acrolein, acrylic acid, methacrolein, methacrylic acid, maleic acid, fumaric acid, maleic anhydride, methyl vinyl ketone, crotonaldehyde, and crotonic acid are generated and the concentration thereof in the product gas increases, which may cause various adverse effects. Specifically, the unsaturated carbonyl compounds are dissolved in an absorption solvent, for example, which can be circularly used in the step C, consequently impurities accumulate in the absorption solvent, for example, and the precipitation of deposits in each structural member is likely to be induced.

Example methods of limiting the concentration of the unsaturated carbonyl compound within a specified range in the oxidative dehydrogenation reaction include a method of adjusting the reaction temperature of the oxidative dehydrogenation. Furthermore, by adjusting the reaction temperature, the concentration of molecular oxygen at the gas outlet of the reactor can be limited within a specified range as well. Specifically, the reaction temperature of the oxidative dehydrogenation is preferably 300° C. or higher and 400° C. or lower, and more preferably 320° C. or higher and lower than 380° C.

By limiting the reaction temperature within the above-described range, coking (precipitation of solid carbon) in the metal oxide catalyst can be suppressed, and the concentration of the unsaturated carbonyl compound in the product gas can be limited within a specified range. Furthermore, the concentration of molecular oxygen at the gas outlet of the reactor can be limited within a specified range as well.

In contrast, when the reaction temperature is excessively low, the conversion rate of n-butene is liable to decrease. Furthermore, the concentration of the unsaturated carbonyl compound increases, and impurities tend to accumulate in the absorption solvent, for example, or coking in the metal oxide catalyst tends to occur.

Preferable specific examples of the method of adjusting the reaction temperature include a method of controlling the catalyst layer temperature so as to be constant through appropriate cooling of the reactor by removing heat, for example, with use of a heat medium (specifically, dibenzyltoluene, nitrite, for example).

### Product Gas

The product gas contains a by-product, an unreacted feedstock gas, unreacted molecular oxygen, inert gases, and water (water vapor), for example, together with 1,3-butadiene as the target product of the oxidative dehydrogenation reaction between the feedstock gas and molecular oxygen.

Examples of by-products include acetaldehyde, benzaldehyde, acetophenone, benzophenone, fluorenone, anthraquinone, phthalic acid, tetrahydrophthalic acid, isophthalic acid, terephthalic acid, methacrylic acid, phenol, and benzoic acid in addition to the above-described unsaturated carbonyl compound having 3 to 4 carbon atoms.

In the product gas discharged out from the reactor, the concentration of molecular nitrogen is preferably 35% by volume or higher and 90% by volume or lower, and more preferably 45% by volume or higher and 80% by volume or lower. The concentration of water (water vapor) is preferably 5% by volume or higher and 60% by volume or lower, and more preferably 8% by volume or higher and 40% by volume or lower. The concentration of butadiene is preferably 2% by volume or higher and 15% by volume or lower, and more preferably 3% by volume or higher and 10% by volume or lower. The concentration of n-butene is preferably 0% by volume or higher and 2% by volume or lower, and more preferably 0.1% by volume or higher and 1.8% by volume or lower. The above-stated concentrations are all based on 100% by volume of the product gas.

When the concentration of each component in the product gas is within the above-described range, the efficiency of butadiene purification to be carried out in the subsequent steps can be improved, and side reactions of butadiene occurring during the purification can be suppressed, whereby the energy consumption in producing butadiene can be further reduced.

### Step B

In the step B, the product gas obtained in the step A is cooled. In the step B, the cooling of the product gas from the step A is usually conducted in a quenching tower 2 and a heat exchanger 3 as shown in FIG. 1.

Specifically, the product gas from the step A, that is, the product gas discharged out from the reactor 1 is sent to the quenching tower 2 via the pipe 101, then cooled in the quenching tower 2, thereafter sent to the heat exchanger 3 via a pipe 104, and further cooled in the heat exchanger 3. The product gas thus cooled in the quenching tower 2 and in the heat exchanger 3 exits out from the heat exchanger 3 and enters into a pipe 105.

### Quenching Tower

The quenching tower 2 has a configuration in which the product gas from the step A is brought into countercurrent contact with cooling medium so that the product gas from the step A is cooled to a temperature of, for example, approximately 30° C. or higher and 90° C. or lower; and in the lower section of the tower is provided a gas inlet configured to introduce the product gas from the step A; and in the upper section of the tower is provided a medium inlet configured to introduce the cooling medium. To the gas inlet is connected the pipe 101 having one end connected to the gas outlet of the reactor 1; and to the medium inlet is connected a pipe 102. At the top of the quenching tower 2 is provided a gas outlet configured to discharge the product gas that has been cooled with the cooling medium; and at the bottom of the tower is provided a medium outlet configured to discharge the cooling medium that has been brought into contact (countercurrent contact) with the product gas from the step A. To the gas outlet is connected the pipe 104; and to the medium outlet is connected a pipe 103.

By the countercurrent contact describe above, the product gas from the step A is purified. Specifically, there is removed some portion of by-product contained in the product gas from the step A.

In the quenching tower 2, water or an alkaline aqueous solution, for example, is used as the cooling medium.

The temperature of the cooling medium (temperature at the medium inlet) is appropriately determined depending on cooling temperature, but is preferably 10° C. or higher and 90° C. or lower, more preferably 20° C. or higher and 70° C. or lower, and particularly preferably 20° C. or higher and 40° C. or lower.

In the quenching tower 2 in operation, the temperature inside the quenching tower 2 is preferably 10° C. or higher and 100° C. or lower, and more preferably 20° C. or higher and 90° C. or lower.

The pressure of the quenching tower 2 (specifically, the pressure at the gas outlet of the quenching tower 2) in operation, that is, the pressure of the step B is preferably equal to or lower than the pressure of the step A.

Specifically, the difference between the pressure of the step B and the pressure of the step A, that is, the value obtained by subtracting the pressure of the step B from the pressure of the step A is preferably 0 MPaG or higher and 0.05 MPaG or lower, and more preferably 0.01 MPaG or higher and 0.04 MPaG or lower.

By limiting the pressure difference between the step A and the step B within the above-described range, the by-product in the product gas from the step A is promoted to condensate and dissolute in the cooling medium in the quenching tower 2, and as a result, there can be further reduced the concentration of by-product (specifically, the concentration of ketones and aldehydes to be described later) in the product gas discharged out from the quenching tower 2.

The product gas discharged out from the quenching tower 2 contains n-butene, molecular oxygen, inert gases, and water (water vapor) together with butadiene, and may contain ketones and aldehydes, for example, as by-products.

The ketones and aldehydes are at least one compound selected from the group consisting of methyl vinyl ketone, acetaldehyde, acrolein, methacrolein, crotonaldehyde, benzaldehyde, acetophenone, and benzophenone.

In the product gas discharged out from the quenching tower 2, the concentration of molecular nitrogen is preferably 60% by volume or higher and 94% by volume or lower and more preferably 70% by volume or higher and 90% by volume or lower. The concentration of n-butene is preferably 0% by volume or higher and 2% by volume or lower, and more preferably 0.1% by volume or higher and 1.8% by volume or lower. The concentration of butadiene is preferably 2% by volume or higher and 15% by volume or lower, and more preferably 3% by volume or higher and 10% by volume or lower. The concentration of water (water vapor) is preferably 5% by volume or higher and 60% by volume or lower, and more preferably 10% by volume or higher and 45% by volume or lower. The concentration of ketones and aldehydes is preferably 0% by volume or higher and 0.3% by volume or lower, and more preferably 0.05% by volume or higher and 0.25% by volume or lower.

The cooling medium that has been discharged out from the quenching tower 2 and has been in contact with the product gas usually contains a by-product, specifically an organic acid, in the product gas from the step A, which has condensed or dissolved in the cooling medium in the quenching tower 2.

The organic acid contained in the cooling medium discharged out from the quenching tower 2 is at least one compound selected from the group consisting of maleic acid, fumaric acid, acrylic acid, phthalic acid, benzoic acid, crotonic acid, tetrahydrophthalic acid, isophthalic acid, terephthalic acid, methacrylic acid, and phenol.

In the cooling medium discharged out from the quenching tower 2, the concentration of the organic acid is preferably 0% by mass or higher and 7% by mass or lower, and more preferably 1% by mass or higher and 6% by mass or lower. When the concentration of the organic acid is excessively high, the load on the wastewater treatment is liable to increase.

### Heat Exchanger

As the heat exchanger 3, there is appropriately used a heat exchanger with which the product gas discharged out from the quenching tower 2 can be cooled to room temperature (10° C. or higher and 35° C. or lower).

In the example of FIG. 1, to the gas inlet of the heat exchanger 3 is connected the pipe 104 having one end connected to the gas outlet of the quenching tower 2, and to the gas outlet of the heat exchanger 3 is connected the pipe 105.

The pressure of the heat exchanger 3 (specifically, the pressure at the gas outlet of the heat exchanger 3) in operation is preferably equal to the pressure of the quenching tower 2 (the pressure at the gas outlet of the quenching tower 2) in the operation.

In the product gas discharged out from the heat exchanger 3, the concentration of molecular nitrogen is preferably 60% by volume or higher and 94% by volume or lower, and more preferably 70% by volume or higher and 85% by volume or lower. The concentration of n-butene is preferably 0% by volume or higher and 2% by volume or lower, and more preferably 0.1% by volume or higher and 1.8% by volume or lower. The concentration of butadiene is preferably 2% by volume or higher and 15% by volume or lower, and more preferably 3% by volume or higher and 10% by volume or lower. The concentration of ketones and aldehydes is preferably 0% by volume or higher and 0.3% by volume or lower, and more preferably 0.05% by volume or higher and 0.25% by volume or lower.

When the concentration of each component in the product gas that has been cooled in the step B is within the above-described range, the efficiency of butadiene purification in the subsequent steps can be improved, and side reactions occurring in the solvent removing step can be suppressed, whereby the energy consumption in producing butadiene can be further reduced.

### Step C

In a step C, by selective absorption into an absorption solvent, 1,3-butadiene is separated from the product gas passed through the step B. Specifically, in the step C, by selective absorption into an absorption solvent, the product gas passed through the step B is separated (coarsely separated) into inert gases and 1,3-butadiene. In the step C, by selective absorption into an absorption solvent, the product gas passed through the step B is preferably separated (coarsely separated) into molecular oxygen and inert gases, and a 1,3-butadiene-containing gas. The term “1,3-butadiene-containing gas” refers to a gas that contains at least butadiene and n-butene (unreacted n-butene), which is to be absorbed in an absorption solvent.

### Absorption Tower

In the step C, the separation of the product gas passed through the step B is conducted in an absorption tower 4 as shown in FIG. 1. In the absorption tower 4, at the lower section of the tower is provided a gas inlet configured to introduce the product gas passed through the step B; at the upper section of the tower is provided a solvent inlet configured to introduce an absorption solvent; at the bottom of the tower is provided a liquid outlet configured to discharge the absorption liquid that has absorbed a gas (specifically, the 1,3-butadiene-containing gas); and at the top of the tower is provided a gas outlet configured to discharge a gas (specifically, molecular oxygen and inert gases) that has not been absorbed in the absorption solvent. To the gas inlet is connected the pipe 105 having one end connected to the gas outlet of the heat exchanger 3; to the solvent inlet is connected a pipe 106; to the liquid outlet is connected a pipe 113; and to the gas outlet is connected a pipe 107.

Specifically describing the step C, the product gas passed through the step B, that is, the product gas discharged out from the heat exchanger 3 is sent to the absorption tower 4 via the pipe 105, and coincident with this, the absorption solvent is supplied to the absorption tower 4 via the pipe 106. In this way, the product gas passed through the step B is brought into countercurrent contact with the absorption solvent, and the 1,3-butadiene-containing gas in the product gas passed through the step B is selectively absorbed in the absorption solvent, whereby the gas is coarsely separated into the 1,3-butadiene-containing gas, and the molecular oxygen and inert gases. Subsequently, the absorption solvent that has absorbed the 1,3-butadiene-containing gas exits from the absorption tower 4 and enters into the pipe 113, while the molecular oxygen and inert gases that have not been absorbed in the absorption solvent exit from the absorption tower 4 and enter into the pipe 107.

In the absorption tower 4 in operation, the temperature inside the absorption tower 4 is not particularly limited, but with increasing temperature inside the absorption tower 4, the molecular oxygen and inert gases are less likely to be absorbed into the absorption solvent, and in contrast, with decreasing temperature inside the absorption tower 4, the absorption efficiency of hydrocarbons such as butadiene (1,3-butadiene-containing gas) into the absorption solvent is likely to increase. In view of the productivity of butadiene, the temperature inside the absorption tower 4 is preferably 0° C. or higher and 60° C. or lower, and more preferably 10° C. or higher and 50° C. or lower.

The pressure of the absorption tower 4 (specifically, the pressure at the gas outlet of the absorption tower 4) in operation, that is, the pressure of the step C is preferably equal to the pressure of the step B or less than the pressure of the step B.

Specifically, the difference between the pressure of the step C and the pressure of the step B, that is, the value obtained by subtracting the pressure of the step C from the pressure of the step B is preferably 0 MPaG or higher and 0.05 MPaG or lower, and more preferably 0.01 MPaG or higher and 0.04 MPaG or lower.

By limiting the pressure difference between the step B and the step C within the above-described range, the absorption of butadiene (1,3-butadiene-containing gas) into the absorption solvent in the absorption tower 4 can be promoted, and as a result, the consumed usage of the absorption solvent can be reduced, and the energy consumption can be reduced.

In particular, it is preferable that the steps A to C are held at an equal pressure, or that step A, step B, and step C each are held at a pressure decreasing in this order. When the pressure of the step B and the pressure of the step C each are equal to or lower than the pressure in the preceding step, the reaction efficiency of oxidative dehydrogenation can be improved, and the energy consumption can be reduced.

In the method of producing 1,3-butadiene according to an embodiment of the present invention, the pressure of the step A is held within a specified range and the step B and the step C each are held at a pressure equal to or lower than the pressure in the preceding step, whereby the reaction efficiency of oxidative dehydrogenation can be improved, and it can be reduced the energy consumption required in the step B and its subsequent steps for purifying the product gas obtained in the step A.

### Absorption Solvent

Examples of absorption solvents include those containing an organic solvent as a main component. The wording “containing an organic solvent as a main component” here means that the content ratio of the organic solvent in the absorption solvent is 50% by mass or higher. The lower limit of the content ratio of the organic solvent in the absorption solvent is preferably 70% by mass and more preferably 80% by mass.

The absorption solvent may be used alone or two or more.

Examples of organic solvents as a component of the absorption solvent include: aromatic compounds such as toluene, xylene, and benzene; amide compounds such as dimethylformamide and N-methyl-2-pyrrolidone; sulfur compounds such as dimethylsulfoxide and sulfolane; nitrile compounds such as acetonitrile and butyronitrile; and ketone compounds such as cyclohexanone and acetophenone.

The amount (feed) of absorption solvent used is not particularly limited, but is preferably 10 mass times or more and 100 mass times or less, and more preferably 17 mass times or more and 35 mass times or less, with respect to the total flow rate (mass flow rate) of butadiene and n-butene in the product gas passed through the step B.

Note that the absorption solvent supplied to the step C can be circularly used.

By limiting the amount of absorption solvent used within the above-described range, the absorption efficiency of the 1,3-butadiene-containing gas can be improved. In a case where the amount of absorption solvent used is excessively large, the energy consumption used in the purification of the absorption solvent for its cyclic use tends to increase. In case where the amount of absorption solvent used is excessively small, the absorption efficiency of the 1,3-butadiene-containing gas is prone to decrease.

The temperature of the absorption solvent (temperature at the solvent inlet) is preferably 0° C. or higher and 60° C. or lower, and more preferably 0° C. or higher and 40° C. or lower. By limiting the temperature of the absorption solvent within the above-described range, the absorption efficiency of the 1,3-butadiene-containing gas can be further improved.

### Circulation Step

In a circulation step, the inert gases separated in the step C, specifically, the molecular oxygen and the inert gases are sent as a return gas to the step A. In the circulation step, the molecular oxygen and inert gases from the step C are processed through a solvent recovery tower 5 and a compressor 6.

Specifically, the molecular oxygen and inert gases from the step C, that is, the molecular oxygen and inert gases discharged out from the absorption tower 4 are sent via the pipe 107 to the solvent recovery tower 5 where solvent removal process is conducted, thereafter sent via a pipe 110 to the compressor 6, followed by pressure adjustment process as necessary. The molecular oxygen and inert gases from the step C, which have thus undergone the solvent removal process and the pressure adjustment process, exit from the compressor 6 and enter into the pipe 112 toward the reactor 1.

In the example of FIG. 1, after having been discharged out from the solvent recovery tower 5, the molecular oxygen and inert gases flow through the pipe 110, in this course of flowing, some portions of the molecular oxygen and inert gases are discarded via a pipe 111 communicated with the pipe 110. In this way, by providing the pipe 111 configured to discard some portions of the molecular oxygen and inert gases discharged out from the solvent recovery tower 5, the feed of the return gas to the step A can be adjusted.

### Solvent Recovery Tower

The solvent recovery tower 5 has a configuration in which the molecular oxygen and inert gases from the step C are washed with water or a solvent, in which the molecular oxygen and inert gases undergo a solvent removal process. In the middle of the tower is provided a gas inlet configured to introduce the molecular oxygen and inert gases from the step C, and in the upper section of the tower is provided a washing liquid inlet configured to introduce water or a solvent. To the gas inlet of the solvent recovery tower 5 is connected the pipe 107 having one end connected to the gas outlet of the absorption tower 4; and to the washing liquid inlet is connected a pipe 108. Furthermore, in the solvent recovery tower 5, at the top of the tower is provided a gas outlet configured to discharge the molecular oxygen and inert gases after being washed with water or a solvent; and at the bottom of the tower is provided a washing liquid outlet configured to discharge water or a solvent used for washing the molecular oxygen and inert gases from the step C. To the gas outlet is connected the pipe 110; and to the washing liquid outlet is connected a pipe 109.

In the solvent recovery tower 5, the absorption solvent contained in the molecular oxygen and inert gases from the step C is removed, and the removed absorption solvent, together with water or a solvent used for washing, exits out from the washing liquid outlet and enters into the pipe 109 and is recovered via the pipe 109. The molecular oxygen and inert gases from the step C, which have been subjected to the solvent removal process, exit from the gas outlet of the solvent recovery tower 5 and enter into the pipe 110.

Examples of the solvent that can be used in the solvent recovery tower 5 include the absorption solvents exemplified in the step C.

In addition, in the solvent recovery tower 5 in operation, the temperature inside the solvent recovery tower 5 is not particularly limited, but is preferably 0° C. or higher and 80° C. or lower, and more preferably 10° C. or higher and 60° C. or lower.

### Compressor

As a compressor 6, it is appropriately used a compressor with which the pressure of the molecular oxygen and inert gases from the solvent recovery tower 5 can be increased as necessary to the pressure required in the step A.

In the example of FIG. 1, to the gas inlet of the compressor 6 is connected the pipe 110 having one end connected to the gas outlet of the solvent recovery tower 5; and to the gas outlet is connected the pipe 112.

In a case where the pressure of the step C is lower than the pressure of the step A, the compressor 6 increases the pressure by the pressure difference in accordance with the pressure difference between the step C and the step A. In a case where the pressure is risen at the compressor 6, the amount of pressure rising is usually small, and thus the electric energy consumption of the compressor remains small.

In the molecular oxygen and inert gas discharged out from the compressor 6, that is, in the return gas, the concentration of molecular nitrogen is preferably 87% by volume or higher and 97% by volume or lower, and more preferably 90% by volume or higher and 95% by volume or lower. The concentration of molecular oxygen is preferably 1% by volume or higher and 6% by volume or lower, and more preferably 2% by volume or higher and 5% by volume or lower.

### Solvent Removing Step

In a solvent removing step, the absorption solvent having absorbed the 1,3-butadiene-containing gas, which is obtained in the step C, is subjected to solvent separation process. That is, the absorption solvent is separated from the absorption liquid from the step C, thereby giving a gas flow of the 1,3-butadiene-containing gas. In the solvent removing step, as shown in FIG. 1, the absorption liquid is separated into the 1,3-butadiene-containing gas and the absorption solvent in a solvent removing tower 7.

Specifically, the absorption liquid from the step C, that is, the absorption solvent discharged out from the absorption tower 4, which has absorbed the 1,3-butadiene-containing gas, is sent to the solvent removing tower 7 via the pipe 113 and subjected to solvent separation process. In the solvent removing tower 7, the 1,3-butadiene-containing gas and the absorption solvent are separated by distillation.

### Solvent Removing Tower

The solvent removing tower 7 has a configuration in which the absorption liquid obtained in the step C is separated by distillation whereby solvent separation process is conducted. In the middle of the tower is provided a liquid inlet configured to introduce the absorption liquid from the step C; at the top of the tower is provided a gas outlet configured to discharge the 1,3-butadiene-containing gas separated from the absorption liquid; and at the bottom of the tower is provided a solvent outlet configured to discharge the absorption solvent separated from the absorption liquid. To the liquid inlet is connected the pipe 113 having one end connected to the liquid outlet of the absorption tower 4; to the gas outlet is connected a pipe 115; and to the solvent outlet is connected a pipe 114.

In the solvent removing tower 7, the absorption liquid from the step C is separated into the 1,3-butadiene-containing gas and the absorption solvent, and the 1,3-butadiene-containing gas exits out from the gas outlet and enters into the pipe 115; and the absorption solvent exits out from the solvent outlet and enters into the pipe 114.

The pressure inside the solvent removing tower 7 is not particularly limited, but is preferably 0 MPaG or higher and 1.0 MPaG or lower, more preferably 0.03 MPaG or higher and 1.0 MPaG or lower, and still more preferably 0.2 MPaG or higher and 0.6 MPaG or lower.

In the solvent removing tower 7 in operation, the temperature at the bottom of the solvent removing tower 7 is preferably 80° C. or higher and 190° C. or lower, and more preferably 100° C. or higher and 180° C. or lower.

### EXAMPLES

Hereinafter, specific examples of the present invention will be described, but the present invention is not limited to these examples.

The gas composition analysis method, methyl vinyl ketone analysis method, ketones and aldehydes analysis method, and organic acid analysis method used are as follows.

The gas composition analysis was conducted by gas chromatography under the conditions shown in Table 1 below. Water (water vapor (H₂O)) was calculated by adding up the amount of water obtained by water-cooled trap at the time of gas sampling.

TABLE 1
Summary of gas composition analysis

| | | |
|---|---|---|
| Gas species | 1,3-butadiene, n-butene | N₂, O₂, COx, H₂O |
| Model | GC-14B (manufactured | GC-14B (manufactured |
| | by SHIMADZU | by SHIMADZU |
| | CORPORATION) | CORPORATION) |
| Detector | FID | TCD |
| Column | TC-BOND Alumina/Na₂SO₄ | WG-100 |
| | 0.53 mm I.D. × 30 m df = | 6.35 mm I.D. × 1.8 m |
| | 10 μm (manufactured by | (manufactured by |
| | GL Sciences Inc.) | GL Sciences Inc.) |
| Carrier gas | N₂ 40 ml/min | He 50 ml/min |
| Temperature | | |
| Injection | 200° C. | 60° C. |
| Detector | 250° C. | 80° C. |
| Column | 60° C. 5 min → 135° C. | 50° C. |
| | (5° C./min) → 185° C. | |
| | (15° C./min) | |

The analyses of methyl vinyl ketone, ketones and aldehydes, and organic acids were conducted by liquid chromatography under the conditions shown in Table 2 below.

TABLE 2

| | |
|---|---|
| Model | LC-2000 Plus (manufactured by JASCO Corporation) |
| Detector | UV (210 nm, 230 nm) |
| Column | TSKgel ODS - 100 V 5 μm |
| | 4.6 mm ID × 15 cm |
| | (manufactured by Tosoh Corporation) |
| Eluent | Acetonitrile/Phosphoric acid aqueous solution |
| | 0.8 ml/min |
| Column oven | 40° C. |

### Example 1

According to the flow diagram of FIG. 1, 1,3-butadiene was produced from a feedstock gas containing 1-butene and 2-butene through the following steps A, B, and C, circulation step, and solvent removing step.

The feedstock gas used was a gas containing 1-butene and 2-butene, in which the ratio of 2-butene to the total of 1-butene and 2-butene was 87% by volume based on 100% by volume of the total of 1-butene and 2-butene.

### Step A

A metal oxide catalyst was filled into a reaction tube (inner diameter: 21.2 mm; outer diameter: 25.4 mm) such that each catalyst layer had a length of 1.5 m, then 77 reaction tubes having the metal oxide catalyst filled were placed in parallel in a cylindrical reactor 1. In this case, the reactor 1 is a shell-and-tube heat exchanger reactor including 77 reaction tubes, in each of whose upper section is filled the metal oxide catalyst so as to be 600 g/tube. A mixed gas having a volume ratio (n-butene/O₂/N₂/H₂O) of 1/1.5/16.3/1.2 was fed to the reactor 1 at a GHSV of 2000 h⁻¹, and thereby causing an oxidative dehydrogenation reaction of the feedstock gas with the molecular oxygen-containing gas under the condition of a reaction temperature of 320 to 350° C. to give a product gas that contains 1,3-butadiene. In this case, the gas inlet was provided at the bottom of the reactor 1, the gas outlet was provided at the top of the reactor 1, and the mixed gas was introduced from the gas inlet (at the bottom) of the reactor 1. The pressure of the step A, that is, the pressure at the gas inlet of the reactor 1 was 0.1 MPaG. The actual volumetric GHSV of the mixed gas was 2150 h⁻¹.

In the step A, as the metal oxide catalyst was used spherical silica on which an oxide of a compound represented by the compositional formula of Mo₁₂Bi₅Fe_{0.5}Ni₂Co₃K_{0.1}Cs_{0.1}Sb0.2 was supported at a ratio of 20% of the total volume of the catalyst.

The mixed gas is a mixture of the feedstock gas and the return gas (molecular oxygen and inert gases), and optionally air as a molecular oxygen-containing gas, molecular nitrogen as an inert gas, and water (water vapor) are additionally mixed thereto so its composition as to be adjusted.

### Step B

The product gas discharged out from the reactor 1 was introduced into the quenching tower 2, and then cooled to 76° C. while being in countercurrent contact with water as a cooling medium, followed by further cooling to 30° C. in the heat exchanger 3. The pressure of the step B, that is, the pressure at the gas outlet of the quenching tower 2 was 0.1 MPaG, and the pressure at the gas outlet of the heat exchanger 3 was also 0.1 MPaG.

In the product gas discharged out from the heat exchanger 3, the concentration of methyl vinyl ketone was 0.008% by volume (80 ppm by volume), and the concentration of ketones and aldehydes was 0.08% by volume (800 ppm by volume).

The concentration of the organic acid in the water that had discharged out from the quenching tower 2 and had come into contact with the product gas discharged out from the reactor 1 was 2.5% by mass.

### Step C

The product gas discharged out from the heat exchanger 3 (hereinafter also referred to as “cooled product gas”) was fed from the gas inlet in the lower section of the absorption tower 4 (outer diameter: 152.4 mm, height: 7800 mm, material: stainless steel of SUS 304) having regular packings arranged therein, and an absorption solvent containing 95% by mass or more of toluene was fed at 10° C. from the solvent inlet in the upper section of the absorption tower 4. The feed of the absorption solvent was 33 times in mass as large as the total flow rate (mass flow rate) of butadiene and n-butene in the cooled product gas. The pressure of the step C, that is, the pressure at the gas outlet of the absorption tower 4 was 0.1 MPaG.

### Circulation Step

The gas discharged out from the absorption tower 4 was washed with water or the solvent in the solvent recovery tower 5 to remove a small amount of the absorption solvent contained in the gas. The gas from which the absorption solvent had been thus removed was discharged out from the solvent recovery tower 5, some portions thereof were discarded, and most of the remaining portion thereof was sent to the compressor 6. In the compressor 6, the pressure of the gas from the solvent recovery tower 5 was increased through pressure adjusting process. In this way, the absorption solvent was removed, and the pressurized gas was discharged out from the compressor 6 and then returned to the reactor 1.

In the gas discharged out from the compressor 6, the concentration of molecular nitrogen was 94% by volume, and the concentration of molecular oxygen was 3% by volume. The gas discharged out from the compressor 6 contained 3% by volume of impurities (specifically, carbon monoxide, carbon dioxide, for example).

### Solvent Removing Step

The liquid discharged out from the absorption tower 4 was fed to the solvent removing tower 7, and the 1,3-butadiene-containing gas was separated from the absorption solvent by distillation to give a gas, this resultant gas is discharged out from the solvent removing tower 7 and cooled in a condenser to yield a 1,3-butadiene-containing gas. In addition, some portion of the liquid discharged out from the solvent removing tower 7 was sent to a reboiler, and an effluent liquid heated in the reboiler, that is, an absorption solvent was also obtained.

After the production step described above had been carried out over 96 hours, the replacement operation of the catalyst layer in reactor 1 was conducted. In the reaction in which the mixed gas containing the feedstock gas and molecular oxygen was introduced from the bottom of reactor 1, only the catalysts in the upper layer portion (20% of upper section) of 77 reaction tubes was needed to be replaced, and the total working time required for the replacement (from the removal of the catalyst to refilling) was 16 hours.

### Control Example 1

There was prepared a reactor 1′ (inner diameter: 21.2 mm; outer diameter: 25.4 mm) in which a metal oxide catalyst was filled such that the catalyst layer had a length of 1.5 m. In this case, the reactor 1′ is a shell-and-tube heat exchanger reactor including 77 reaction tubes, in each of whose lower sections was filled a metal oxide catalyst so as to be 600 g/tube. The same procedure as in Example 1 was carried out except that the gas inlet was provided at the top of the reactor 1′ and the gas outlet was provided at the bottom of the reactor 1′, and the mixed gas was introduced from the gas inlet (at the top) of the reactor 1′.

In the reaction in which the mixed gas containing the feedstock gas and molecular oxygen was introduced from the top of the reactor 1′, all the catalyst layers in the 77 reaction tubes were needed to be replaced, and the total working time required for the replacement (from the removal of the catalyst to refilling) was 40 hours.

TABLE 3

| | | Control |
|---|---|---|
| | Example 1 | Example 1 |
| GHSV [h⁻¹] | 2000 | 2000 |
| Actual volumetric GHSV [h⁻¹] | 2150 | 2150 |
| Pressure of step A [MPaG] | 0.1 | 0.1 |

| | |
|---|---|
| Volume ratio in mixed gas | 1/1.5/16.3/1.2 |

| n-buten/O₂ /N₂ /H₂O | | |
|---|---|---|
| Pressure of step C [MPaG] | 0.1 | 0.1 |
| The number of reaction tubes (pieces) | 77 | 77 |
| Catalyst length [m] | 1.5 | 1.5 |
| Catalyst amount [g/reaction tube] | 600 | 600 |
| Catalyst removing time [h] | 8 | 8 |
| Catalyst refilling time [h] | 8 | 32 |
| Total working time for catalyst | 16 | 40 |
| replacement [h] | | |

### Reference Signs List

1 Reactor
2 Quenching tower
3 Heat exchanger
4 Absorption tower
5 Solvent recovery tower
6 Compressor
7 Solvent removing tower
100 to 116 Piping

1. A method of producing 1,3-butadiene, comprising:
feeding oxygen and a feedstock gas comprising n-butene into a reactor from a lower section of the reactor provided with a metal atom-containing catalyst, such that a product gas comprising 1,3-butadiene is obtained through oxidative dehydrogenation of the n-butene;
cooling the product gas comprising the 1,3-butadiene; and
separating the 1,3-butadiene from the cooled product gas through a selective absorption into an absorption solvent.

2. The method according to claim 1, wherein the reactor is a shell-and-tube heat exchanger reactor comprising a plurality of reaction tubes in which the metal atom-containing catalyst is filled.

3. The method according to claim 1, wherein the metal atom-containing catalyst is a composite oxide catalyst comprising at least molybdenum and cobalt.

4. The method according to claim 1, wherein the feedstock gas comprises n-butene at a concentration of 40% by volume or higher.

5. The method according to claim 1, wherein the feeding is held at a pressure of 0 to 0.4 MPaG, and the feeding, the cooling and the separating are held at an equal pressure.

6. The method according to claim 1, wherein the feeding is held at a pressure of 0 to 0.4 MPaG, and the feeding, the cooling and the separating are each held at a pressure decreasing in this order.
